(19) United States
(12) Patent Application Publication
(10) Pub. No.: US 2022/0054041 A1
(43) Pub. Date: Feb. 24, 2022

(54) LOOP-BASED MEASURING DEVICE

(71) Applicant: CARLO MENON, Coquitlam (CA)

(72) Inventors: CARLO MENON, Coquitlam (CA); MOHAMMAD TAVASSOLIAN, Coquitlam (CA); ASTRID GARCIA PATINO, Burnaby (CA)

(21) Appl. No.: 16/995,815

(22) Filed: Aug. 18, 2020

(51) Int. Cl. A61B 5/11; A61B 5/107; A41H 1/02; G01B 7/02; A61B 5/00; G06N 20/00

(52) U.S. Cl. CPC A61B 5/11; A61B 5/1072; A41H 1/02; A61B 2505/09; A61B 5/6804; G06N 20/00; A63B 2220/836; G01B 7/02

(57) ABSTRACT

The present invention will provide a measuring device embedded within an elastic garment that will provide detect bodily movement by deforming a conductive material and measuring the change in electrical quantities within the conductive material as it deforms. Furthermore, the present invention will provide a measuring device configured to incorporate machine learning algorithms to estimate physiological, kinematic, dynamic, psychological, physical, biological, or other health parameters based upon the variations in electrical quantities within the conductive material as it deforms. This is accomplished through an elastic textile, a conductive element embedded within the elastic textile, and an electronic device configured to measure electrical quantities of the conductive element. These elements work in conjunction to detect and monitor movement in the human body.

### CROSS-REFERENCE TO RELATED APPLICATIONS

This application claims the benefit of U.S. Provisional Patent Application No. 62/887,419, filed on Aug. 15, 2019, and incorporated herein by reference.

### STATEMENT REGARDING FEDERALLY SPONSORED RESEARCH AND DEVELOPMENT

Not Applicable.

### FIELD OF THE INVENTION

This invention relates generally to a device configured to detecting movements of the human body, and more particularly, to a loop-based sensor configured to monitor the electrical properties of a conductive element when said conductive element deforms.

### DISCUSSION OF RELATED ART

Monitoring the movement and physical characteristics of the human body can be extremely beneficial in various medical fields such as preventative medicine, sports medicine, rehabilitation, and physical training. The bending or torsion of one or multiples joins, such as a set of vertebras of the human body, may be of great importance to prevent injuries and for therapeutic purposes. The information collected by measuring and monitoring these movements can accelerate rehabilitation and prevent additional injuries during recovery, as well as provide insight to prevent similar injuries in the future.

A wearable sensor is described in an article by Ravindra Wijesiriwardana, title “Inductive Fiber-Meshed Strain and Displacement Transducers for Respiratory Measuring Systems and Motion Capturing Systems”, published in the IEEE Sensor Journal, Vol. 6, No. 3, in June 2006, pages 571-579. (DOI: 10.1109/JSEN.2006.874488). This article presents the use of electro conductive fibers (polymeric and metallic) in coils configurations. The electro conductive fiber coils structures are made and integrated into the garments by a flatbed-knitting technology. The measurement of a joint angle was result of the calculation of the self-inductance of a single coil or the mutual inductance of multiple coils.

U.S. Pat. No. 9,681,826B2 introduces a method, device and system for measuring a degree of torsion or bending of a human or animal joint, like a knee. The sensor involves an electrically conductive loop that runs from one limb to other and back. The inductance of said sensor is calculated and used as output signal. The method consist in the steps of attaching the sensor to the limbs that form a joint, measuring an output signal of the sensor, and relating the sensor's output signal to a degree of torsion or bending. Said patent mentions the use of accelerometer to trigger an automatic recurring calibration or to measuring the bending or torsion when the human or animal is in rest position.

U.S. Pat. No. 6,341,504B1 presents a fabric for physiological monitoring with one or more stretchable bands. The stretchable bands include at least one conductive wire attached in a curved pattern. When the fabric stretches, the curvature of the conductive wire changes. In consequence of said stretch, the inductance value of the conductive wires varies.

U.S. Pat. No. 7,319,895B2 discloses a garment for medical monitoring, where the data and electrical power are distributed by elastic conductive yarns integrated into the garment. The elastic conductive yarns consist of a conductive yarn in spiral configuration around a non-conductive elastic yarn.

U.S. Pat. No. 8,961,439B2 presents a system for analyzing human gait using textile sensors. The embodiments disclosed an objective of sense gait analysis and posture changes that can be utilized in different fields, such as, rehabilitation, physical training, long-term care, orthopedic and sport medicine, interactive computer games, among others. The system is conform by two elements; a sock sensing system and a processor. The sock sensing system comprises at least one switch, tension sensor, or pressure sensor for detecting posture or movement. The processor is configured to receive signals from the sock sensing system and to analyze a gait parameter.

U.S. Pat. No. 9,858,611B2 introduces a measurement apparatus configured to be worn by the user that indicates the dimension of a part of the body. Said apparatus includes an elastic fabric, one or more conductive fibers, and a controller. The conductive fibers are integrated with the elastic fabric, and when stretched, present a change in an electrical property, which is measured by the controller. The controller also output an indication of a dimension of the part of the body based on the measured change. Said patent describes the application of this technology to find the proper fit when online shopping.

A series of patents disclosing a non-invasive physiological monitoring system (U.S. Pat. Nos. 6,047,203A, 6,551,252B2, 7,670,295B2, 9,462,975B2, 9,750,429B1). Where said system includes several sensors to monitor parameters reflecting pulmonary, cardiac or other organ functions. To monitor different physiological signals of the human body, the system integrates IP sensors, electrodes, a body position sensor, pulse oximeter, a microphone, among others. These patents describe in detail the position and configuration of the sensors used, and sensor's data processing.

While various devices exist which may assist in detecting bodily movement, they are often external devices which are cumbersome to use and do not provide accurate measurements across various parts of the body. Therefore, the need exists for a measuring device embedded within an elastic garment that will provide detect bodily movement by deforming a conductive material and measuring the change in electrical quantities within the conductive material as it deforms. Furthermore, the need exists for a measuring device configured to incorporate machine learning algorithms to estimate physiological, kinematic, dynamic, psychological, physical, biological, or other health parameters based upon the variations in electrical quantities within the conductive material as it deforms. The present invention satisfies these needs.

### SUMMARY OF THE INVENTION

The present invention will provide a measuring device embedded within an elastic garment that will provide detect bodily movement by deforming a conductive material and measuring the change in electrical quantities within the conductive material as it deforms. Furthermore, the present invention will provide a measuring device configured to incorporate machine learning algorithms to estimate physiological, kinematic, dynamic, psychological, physical, biological, or other health parameters based upon the variations in electrical quantities within the conductive material as it deforms. This is accomplished through an elastic textile, a conductive element embedded within the elastic textile, and an electronic device configured to measure electrical quantities of the conductive element. These elements work in conjunction to detect and monitor movement in the human body.

Embodiments of the present invention provide a measurement of metrics and parameter related to a human body, such as joint angle, joint position, posture monitoring, gait monitoring, and various other parameters. Said embodiments can be applied to preventive medicine, rehabilitation, sport medicine, physical training, virtual reality and other fields.

In a first embodiment, the present invention comprises an elastic fabric, at least one conductive element, and a controller. The elastic fabric is configured as a fully or partially tight-fitting garment meant to be worn by the user over at least a part of the body. The conductive elements are attached into the elastic fabric using basic stitches (e.g., straight, zigzag, wave form, stretch stitch, knit stitch, blind hem) to stretch in combination with the elastic fabric over the human body part of interest. The conductive elements may consist of thin copper wire, thread made of a combination of metallic filaments, conductive thread, silver coated thread, conductive and stretchable tube filled with ionic liquids, an elastic thread wrapped with conductive material, among others. As used herein, the term “attached” refers that conductive elements may be sewn, knitted, embedded, attached using adhesives or intermeshed with an elastic textile portion. A controller is connected to measure a change in an electrical property of at least one conductive element in response to stretching of the elastic fabric, and to output a metrics of interest of the corresponding part of the body where the elastic fabric is worn. The output metrics can be wirelessly transmitted to another device (e.g. cellphone, tablet, computer, etc.).

In an alternative embodiment, the elastic fabric may be configured as a tight-fitting garment for upper body, such as a leotard or t-shirt. A single conductive element may be attached on the elastic fabric by sewing them using basic stitches (e.g., straight, zigzag, wave form, stretch stitch, knit stitch, blind hem) such that the conductive element is located in the posterior section of the user.

In a further alternative embodiment, the conductive element may be in a flat coil configuration, where the higher concentration of the coil is covering multiple vertebras of the user's lower back, such that when back flexion is performed, the elastic fabric and the conductive element stretch, resulting in an electrical property change, such as inductance, resistance or capacitance.

In a further alternative embodiment, the controller may be configured to measure the conductive element's electrical property and process it to output indication of the bending angle and/or posture.

In a further alternative embodiment, the elastic fabric may be configured as a tight-fitting garment for lower body, such as a short or leggings. At least one conductive element may be attached on the elastic fabric by sewing them using basic stitches (e.g., straight, zigzag, wave form, stretch stitch, knit stitch, blind hem) such that the conductive element is located in the areas of interest, like knee and hip join.

In a further alternative embodiment, the conductive element may be combined with a stretchable non-conductive filament, said combination is made by coiling the conductive element around a filament that is stretchable and non-conductive. Combined elements may be configured in a flat loop shape and placed over and/or close to the areas of interest (e.g. knee, hip, ankle, etc.). The stretch of the elastic fabric and consequently, the combined elements expansion, result in a change of an electrical property.

In a further alternative embodiment, the controller may be configured to measure the conductive element's electrical property and process it to output indication of at least one gait parameters, for example, step length, stride length, cadence, speed, foot angle, hip angle, progression line, among others.

In a further alternative embodiment, the elastic fabric may be configured to be worn over a single joint, for example elbow or knee. Said embodiments may contain at least one conductive element attached on the elastic fabric. The conductive element may go over the joint, such that a strain on the elastic fabric results in a change in inductance of the conductive element.

In a further alternative embodiment, the conductive elements may be configured in a loop, where said loop is an area encircled by the filament which may be closed or open. Here, the controller may sense changes of an electrical property, such as inductance, said change of electrical property may be correlated with joint movements related to the skin area. The conductive elements' loop may encircle from the proximal tibial epiphysis to the distal femoral epiphysis, said loop may cover partially or completely the mentioned area. A controller may monitor the conductive element's inductance and processes it to output indication of the joint angle, angular velocity, etc.

In a further alternative embodiment, the sensor described earlier can be used to measure the change of human biological parameters, such as Body Mass Index (BMI). Mention sensor produces a known electromagnetic field that can be used to measure the fat of a body based on the degree of perturbation of the electromagnetic field. These perturbations are results of the quantity and volume of electrolytes' distribution in the body where the sensor is attached.

In a further alternative embodiment, the controller incorporates a wireless communication that is configured to output the indication of the desired parameter (e.g. joint angle, posture, gait parameters, etc.) to an external device like a computer, cellphone or table via the wireless communication.

In a further alternative embodiment, the controller may be configured to output the indication of a desired parameter such as; posture, joint angle or gait parameter via wireless communication to an external application installed in an external device. The external application is configured to read the information received from the controller and display a plot with said parameters, as well as, save them in a computer file. Said file may be shared via the available option of the device where the external application is installed, for example, email, messaging apps, cloud storage apps, etc.

In a further alternative embodiment, a threshold may be set in the external application such that when the received parameter exceeds said threshold, the device where the external application is installed will send a notification to the user, for example vibration or a sound.

In a further alternative embodiment, the signals obtained from the sensors may be fed as inputs along with any additional sensor's signal (e.g. IMUs) to a software algorithm, such as machine learning algorithms. The training of the model may use the sensor's raw signal as well as the signals of the complementary sensors to estimate the indication of the desired parameter (e.g. joint angle, posture, gait parameters, etc.). Additionally the input data to the model may be preprocessed using signal filtering techniques, manual feature extraction as well as automatic feature extraction by a third party software library to provide an extra corroboration to the software algorithm or model.

These and other objectives of the present invention will become obvious to those of ordinary skill in the art after reading the following detailed description of the preferred embodiments. It is to be understood that the foregoing general description and the following detailed description are exemplary, and are intended to provide further explanation of the invention as claimed.

### DESCRIPTION OF THE DRAWINGS

FIG. 1 is a rear view of the present invention according to an exemplary embodiment of the invention;

FIG. 2 is a rear view of the present invention for monitoring hip movement according to one embodiment of the present invention;

FIG. 3 is a front view of the present invention for monitoring the thoracic area according to one embodiment of the present invention;

FIG. 4 is a perspective view of the present invention for monitoring the elbow according to one embodiment of the present invention;

FIG. 5 is a perspective view of the present invention for monitoring the ankle according to one embodiment of the present invention;

FIG. 6 is a schematic view of a sensing element according to one embodiment of the present invention;

FIG. 7 is a schematic view of a sensing element according to one embodiment of the present invention;

FIG. 8 is a schematic view of a sensing element according to one embodiment of the present invention;

FIG. 9 illustrates various shape configuration of a sensing element of the present invention;

FIG. 10 is a schematic view of a composite fabric constructed according to one embodiment of the present invention in a patch form;

FIG. 11 is a schematic view of the composite fabric constructed according to one embodiment of the present invention in a patch form.

FIG. 12 is a front perspective view of the present invention according to one embodiment;

FIG. 13 is a rear perspective view therein;

FIG. 14 illustrates various concentric shape configurations of a sensing element of the present invention;

FIG. 15 illustrates various concentric shape configurations of a sensing element of the present invention connected in parallel;

FIG. 16 illustrates various three-dimensional shape configurations of a sensing element of the present invention;

FIG. 17 is a schematic view of the sensing element of the present invention according to one embodiment of the present invention; and

FIG. 18 is a front perspective view of the present invention according to one embodiment of the present invention.

### DETAILED DESCRIPTION OF THE PREFERRED EMBODIMENT

Illustrative embodiments of the invention are described below. The following explanation provides specific details for a thorough understanding of and enabling description for these embodiments. One skilled in the art will understand that the invention may be practiced without such details. In other instances, well-known structures and functions have not been shown or described in detail to avoid unnecessarily obscuring the description of the embodiments.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list.

The embodiments disclosed in the present invention provide apparatus and methods for measuring a joint angle, posture and/or gait parameters, as well as provide feedback about aforementioned parameters. The embodiments described below consist of an elastic fabric configured to be worn by the user with electronics attached to it. When the embodiment is worn by the user, it automatically measures the variables solicited, including posture, angle joint and/or gait parameters. The disclosed embodiments are particularly beneficial in medical fields, such as preventive medicine, sport medicine, rehabilitation and physical training.

In a first embodiment, the present invention is used to monitor the back posture to prevent the occurrence of low back pain and/or improve the user posture by notifying him/her when a harmful or incorrect posture is being held. Furthermore, the present invention can notify the user by sound or vibration. In an alternative embodiment, the notifications can be delivered by wireless communication with an external device, for example a cellphone. Here, the present invention is wirelessly connected to an external device using an external application. The external application, or app, displays the information received by the embodiments, as well as, plotting the information in the screen. It is also possible to generate a computer file and storage it on the phone and/or share it by email, messaging apps, cloud storage apps, among other alternatives available on the cellphone.

In an alternative embodiment, the elastic fabric is configured to be worn over a single joint, for example elbow or knee. Here, the present invention comprises at least one conductive element attached on the elastic fabric. The conductive element goes over the joint such that a strain on the elastic fabric results in a change in an electrical property such as, resistance, capacitance or inductance. The mentioned electrical change is captured by a controller and processed into a desired parameter, for example, joint angle. This is particularly beneficial in fields like sport medicine, rehabilitation, physical training and virtual reality, due to the possibility of measure a joint angle.

In a further alternative embodiment, the elastic fabric with at least one conductive element is implemented in a patch configuration. The patch can be attached in any area of a garment where a measurement of strain is required. As used herein, the term “attached” refers that the elastic fabric may be sewn, knitted, embedded, or paste on a garment meant to be worn by the user.

In a further alternative embodiment, in the present invention may contain a combination of conductive elements, where each conductive element in presence of strain and/or pressure result in a change of different electrical property like inductance, capacitance or resistance. Said electrical property changes can be received and processed by the controller integrated in each measurement apparatus. In a further alternative embodiment, mentioned electrical changes can be an input into a machine learning algorithm to enhance the performance of the model trained to measure a parameter, such as join angle, posture, speed, gait parameters, etc.

In a further alternative embodiment, IMU's can be used to improve the performance of the measurement apparatus discussed in the present invention. Furthermore, embodiments which conductive element's measurement is based on inductance can be used to harvest power due to the Electromagnetic Field (EMF) produced by creation of inductance in the sensor which is a coil.

In the preferred embodiment, the measurement apparatus comprises at least one conductive element, elastic fabric and a controller, which is commonly attached to the elastic fabric or garment. The controller is programmed to read the change of an electrical property when strain and/or pressure is applied to the conductive elements as well as process the data into the desired parameters (e.g. posture, joint angle, gait parameters, etc.). In alternative embodiments, the controller may wirelessly communication with external devices, such as, computer, cellphone, tablet, etc. Additionally, some embodiments are wirelessly connected to an external device using an app. Said app displays the information received by the embodiments, as well as, plotting the information in the screen. It is also possible to generate a computer file and store it on the phone and/or share it through email, messaging apps, cloud storage apps, among other alternatives available on the cellphone.

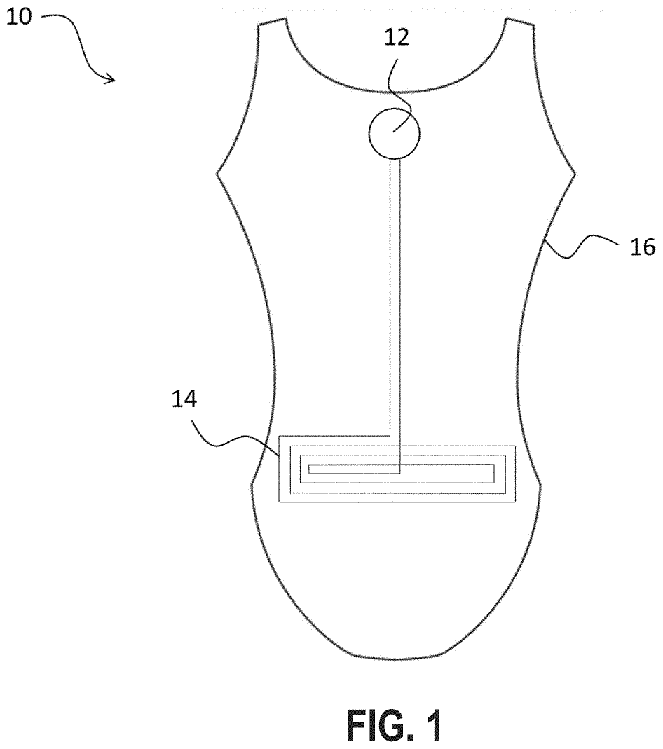

Referring now to FIG. 1, there is a perspective rear view of a smart garment, shown as 10, for use to monitoring the lower back movements of the user. Smart garment 10 may include an elastic textile portion 16, an electronic device 12, at least one conductive element in a flat coil configuration 14.

Smart garment 10 may be adapted to be worn by a user. Sensors 14, which may be positioned at the user's lower back level, and may sense physiological or performance characteristics of the user. Physiological characteristics may be indicative of conditions of the user's body (e.g., respiration). Performance characteristics may be indicative of behavior of the user's body to respect to a parameter of interest (e.g., movement, position, speed). Moreover, sensors 14 may transmit data of the aforementioned characteristics to an electronic device 12 positioned on the upper level of the textile portion 16.

Sensors 14 may consist in a conductive element such as, a thin copper wire or a conductive thread made by a combination of metallic wires, sewn into a textile portion 16 using hand or machine sewing stitches, for example, a zigzag stitch.

Electronic device 12 may contain a microcontroller to read the data sent by sensors 14, a wireless connection device and batteries. Electronic device 12 may receive the data sent by sensors 14 and process it in order to obtain a value of interest or an electrical characteristic of sensors 14 (e.g., angle, velocity, coordinate, distance, voltage, inductance, resistance, capacitance), as well as wireless transmission of the data via to an external device (e.g., computer, cellphone, tablet, etc.)

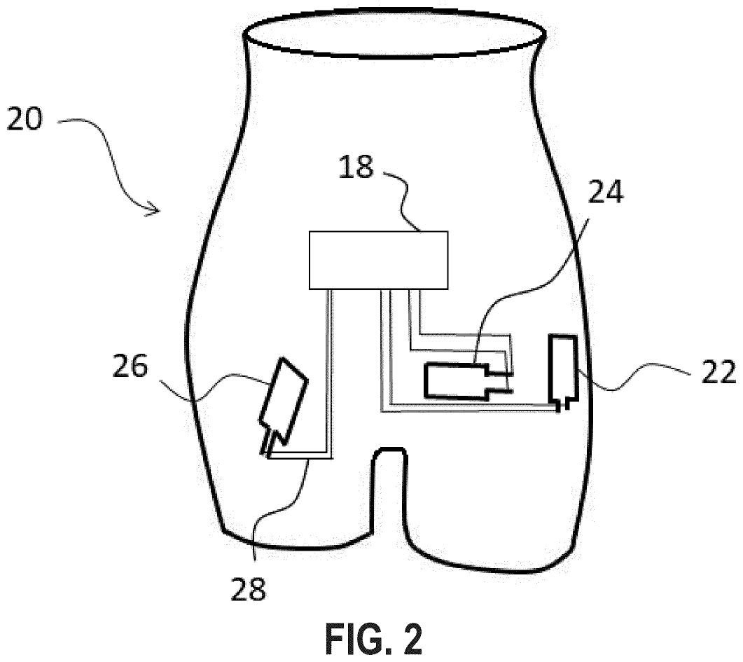

Referring now to FIG. 2, a suggestive position 20 with three flat loop sensors, 22, 24 and 26 are placed on a mannequin representing a human body. The term “flat” refers to the loop placed at the surface of skin/clothes, said loop does not encircle any segment of the human body. Sensor 22 provides information about movement of hip angle without having to encircle the whole hip. Sensors 22, 24 and 26 can be embedded into a garment. Element 28 represents the potentially wiring necessary to connect the aforementioned sensors to a PCB board 18 to read inductance and process the data. Processing can be used to alert the user for special instances or send the processed/unprocessed data to a cloud.

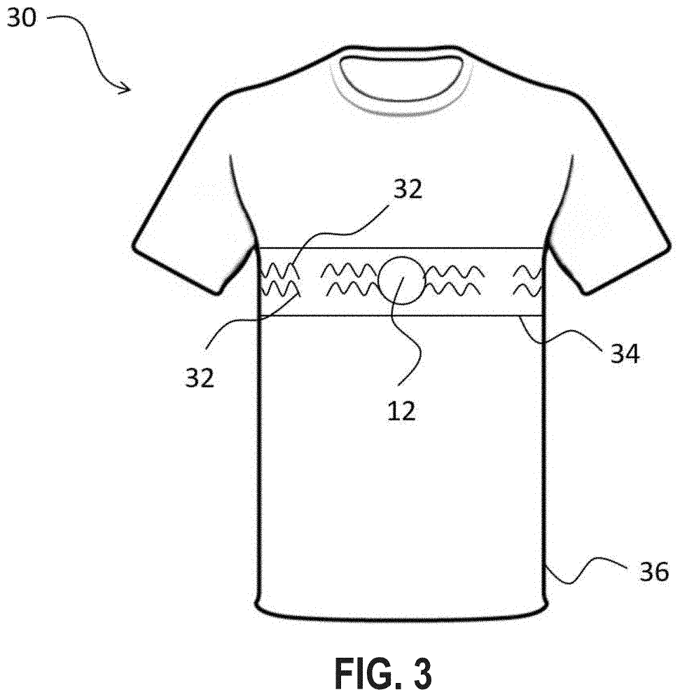

Referring now to FIG. 3, a smart garment 30 is shown for use in monitoring the breathing of the user. The smart garment 30 may include a tight-fitting garment 36, an elastic textile portion 34, an electronic device 12, and at least one sensor 32 located over any area of the textile portion 34.

The smart garment 30 may be adapted to be worn by a user. The elastic textile portion 34 may be located in the thoracic section of the smart garment 30. At least one sensor 32 may be attached on an elastic textile portion 34 in a flat loop configuration using basic stitches (e.g., straight, zigzag, wave form, stretch stitch, knit stitch, blind hem) or others. As used herein, the term “attached” refers that sensors 32 may be sewn, knitted, embedded, or intermeshed with an elastic textile portion 34. Sensors 32 may also sense physiological or performance characteristics of the user and transmit the data to an electronic device 12. Physiological characteristics may be indicative of conditions of the user's body (e.g., respiration). Performance characteristics may be indicative of behavior of the user's body to respect to a parameter of interest (e.g., movement, position, speed).

The electronic device 12 may contain a microcontroller to read the data sent by sensors 32, wireless communication and batteries. The electronic device 12 may receive the data sent by sensors 32 and process it in order to obtain a value of interest (e.g., angle, velocity, coordinate, distance, respiration, heart rate), as well as transmit wirelessly the data to another external electronic device (e.g., computer, cellphone, tablet, etc.)

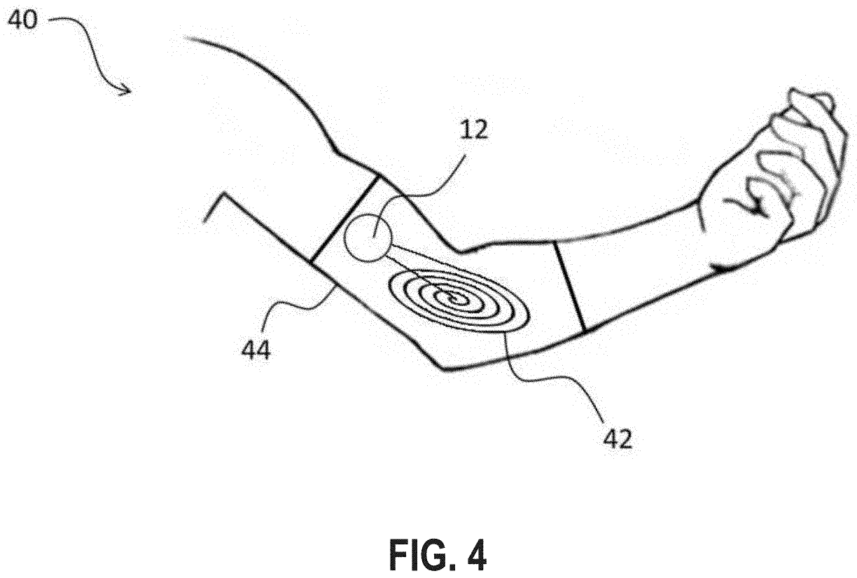

Referring now to FIG. 4, a sensing element 40 is shown with a textile portion 44, at least one sensor 42, and an electronic device 12. The sensing element 40 may be adapted to be worn over a joint by a user. The sensor 42 may be attached on a textile portion 44 in a close loop configuration using basic stitches (e.g., straight, zigzag, wave form, stretch stitch, knit stitch, blind hem) or others. As used herein, the term “attached” refers that sensors 42 may be sewn, knitted, embedded, or intermeshed with an elastic textile portion 44. Sensors 42 may as well, sense the performance characteristics of the user and transmit the data to an electronic device 12. Performance characteristics may be indicative of behavior of the user's body to respect to a parameter of interest (e.g., movement, position, speed).

The electronic device 12 may contain a microcontroller to read the data sent by sensors 42, wireless communication and batteries. The electronic device 12 may receive the data sent by sensors 42 and process it in order to obtain a value of interest (e.g., angle, velocity, coordinate, distance, respiration, heart rate), as well as wirelessly transmit the data to an external device (e.g., computer, cellphone, tablet, etc.)

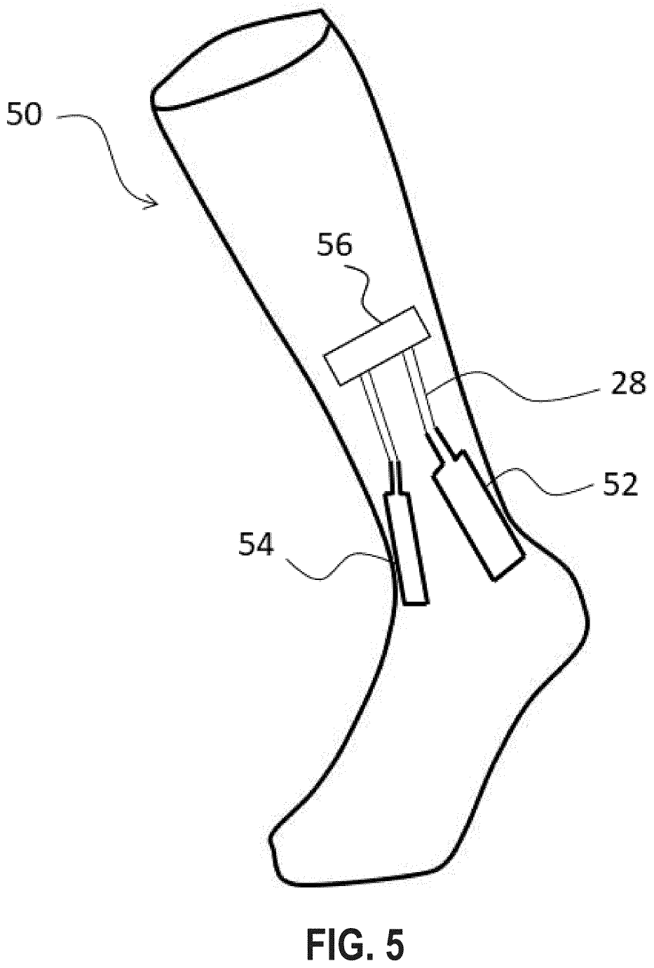

Referring now to FIG. 5, the present invention is positioned on the ankle's dorsiflexion, plantar flexion, eversion, and inversion. The sensors 52 and 54 are sample sensor batch attached to a stretchable socks, using basic stitching, or adhesive techniques. Wiring 28 connects sensors 52 and 54 to a computing unit 56 to read inductance, apply post processing steps and send the data to the cloud or alert the user via a wired/wireless way.

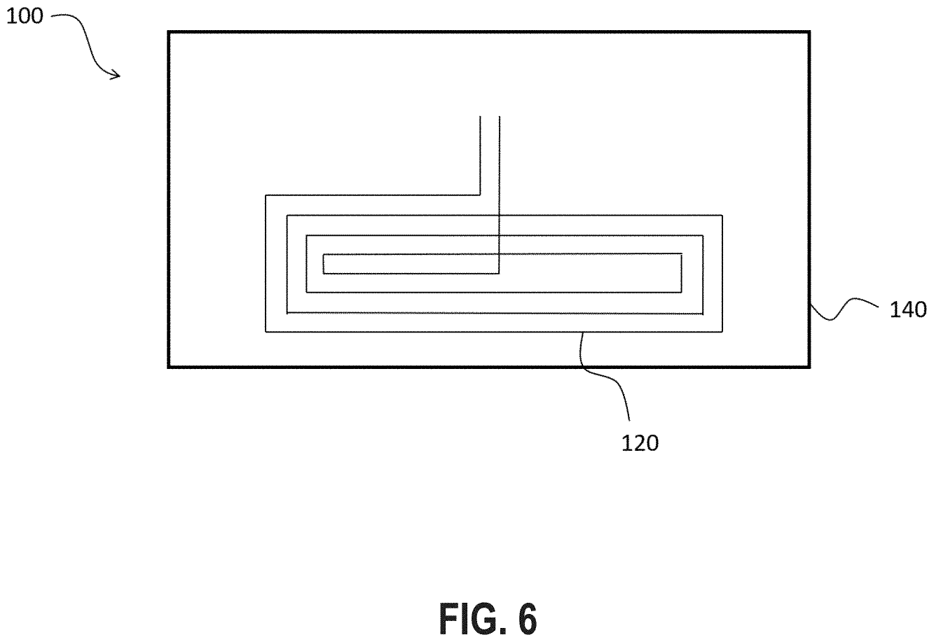

Referring now to FIG. 6, a schematic, detailed view of the sensing element 100 is shown. The sensing element 100 may contain at least one conductive element 120 that can be sewn to a textile portion 140. Conductive element 120 may be stitch in a flat coil configuration using a basic stitch. Sensor 120 may be consist of at least one conductive element, such as a thin copper wire, a conductive metallic thread or a silver coated thread. When sensor 120 is present of a deformation, it may presents a variation in its inductance or resistance value, that later can be interpreted as physiological characteristic (e.g. respiration) or a performance characteristic that may be indicative of behavior of the user's body to respect to a parameter of interest (e.g. movement, angle, position).

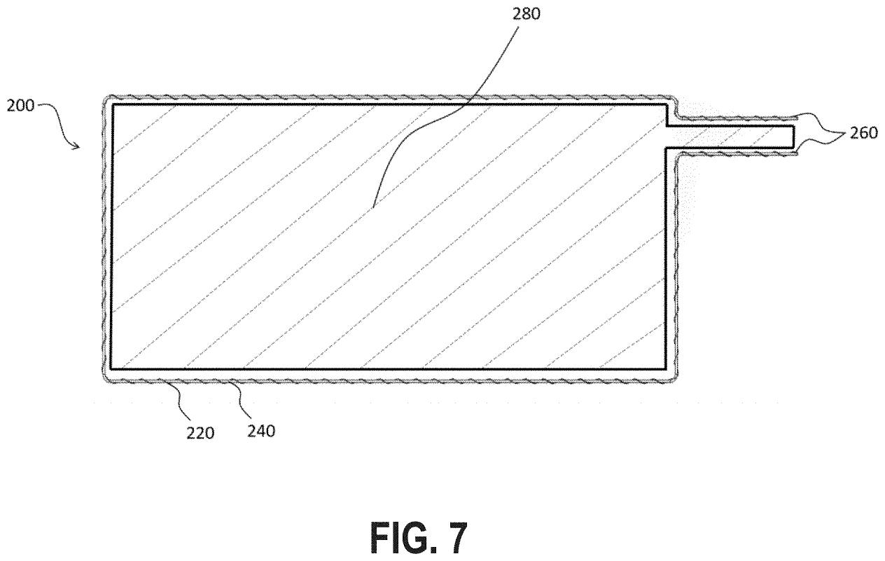

Referring now to FIG. 7, a schematic, detailed view of a sensing element 200 is shown where an open loop of conductive 220 and stretchable material 240 combined such that can tolerate and elongate once a force is applied. The open loop will turn into a closed loop once placed as a part in a proper electronic circuit such as but not limited to LC circuit, using the terminals 260. The skin deformation causes the area 280 encircled by the loop to change, once a force is applied at proper location on the sensor as a result of garment stretching in a direction. As a result of this stretch, the area 280 encircled by the loop changes, affecting the self-inductance of the sensor which can be read by placing it in a proper electronic circuit. Therefore strain can be correlated with self-inductance measured.

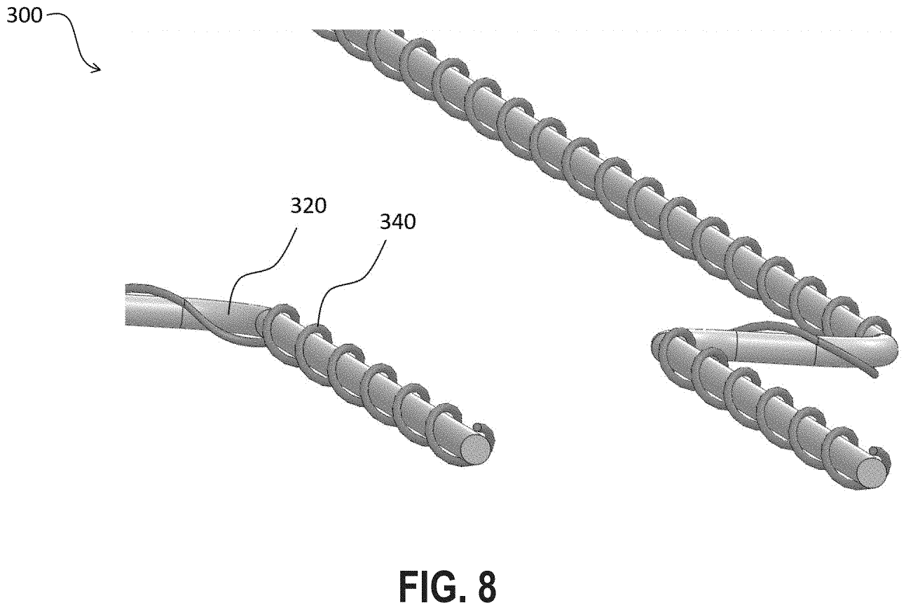

Referring now to FIG. 8, a schematic, detailed view of the sensing element 300 is shown. The sensing element 300 is an example how the conductive material can be combined with stretchable filament, where the conductive material can be a thin copper wire 340 and stretchable filament can be at least a yarn of spandex 320. In this embodiment, the conductive element may be combined with a stretchable non-conductive filament, said combination is made by coiling the conductive element around a filament that is stretchable and non-conductive. Combined elements may be configured in a flat loop shape and placed over and/or close to the areas of interest (e.g. knee, hip, ankle, etc.). The stretch of the elastic fabric and consequently, the combined elements expansion, result in a change of an electrical property.

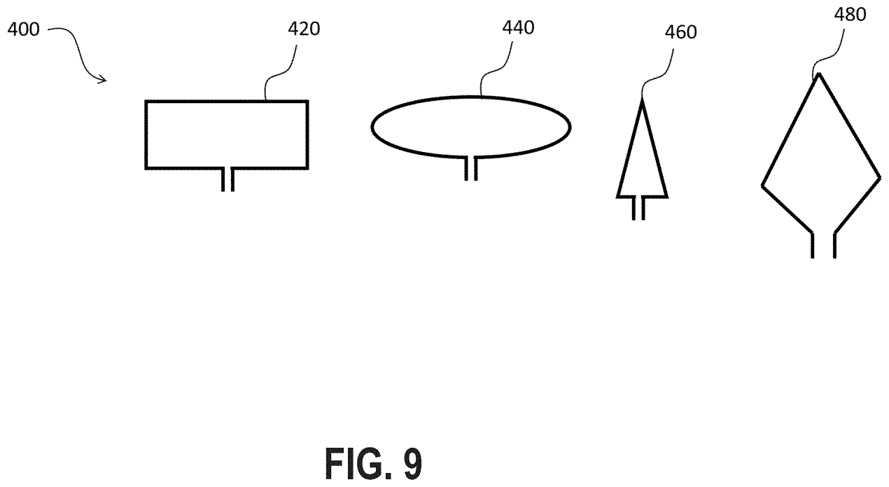

Referring now to FIG. 9, a schematic view 400 is shown of a possible shape loop configurations. The loop can be formed by irregular, closed shapes which may have discontinuous sections, such as a rectangular loop 420, a circular shape 440, triangular shape 460, and generic polygon 480. The loops presented by 400 are fabricated by, for example at least one sensing element 300 (shown in FIG. 8). However, it is possible to increase the number of sensing elements 300 in order to achieve a higher quality signal.

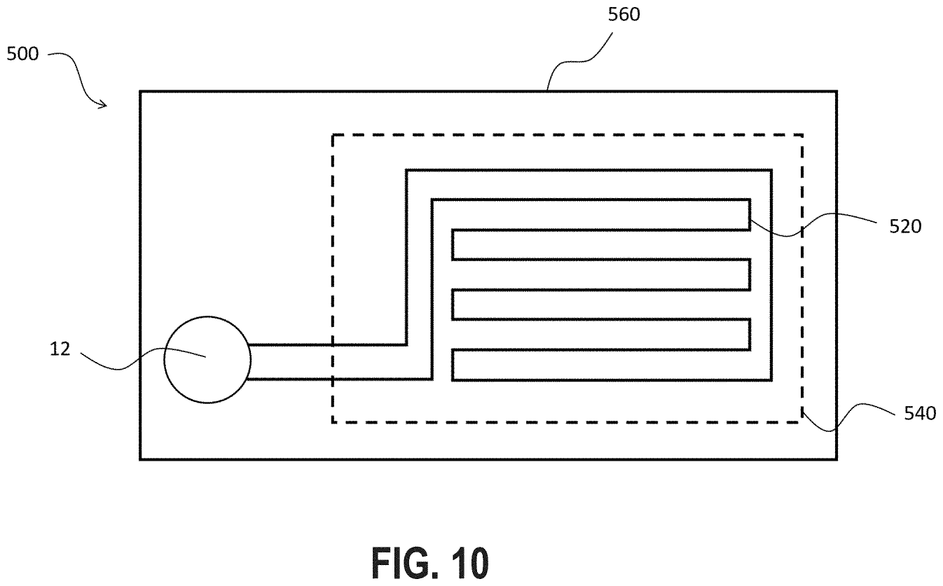

Referring now to FIG. 10, a perspective view of a composite fabric is shown in a patch configuration 500. Composite fabric 500 may include a textile portion 560, a removable stabilizer 540, at least one sensor 250, and an electronic device 12. Patch 500, may be used to integrate in other garments for a desire reading of a physiological or performance characteristics of the user. Patch 500 can be manufactured with different dimension depending on the require application (e.g., measurement of range of motion of a member of a human body, angle, velocity, position, respiration).

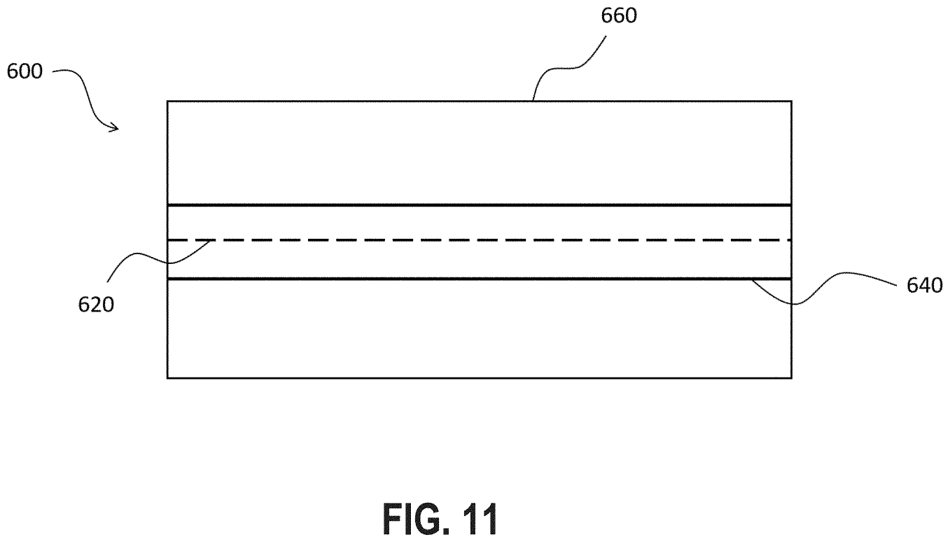

Referring now to FIG. 11, a perspective view of the composite fabric 600 is shown in a patch form according to the present invention. Composite fabric 600 may include a textile portion 660, an elastic component 640, and at least one sensor 620. Sensor 620 may be attached to an elastic component 640 in a close loop configuration using basic stitches (e.g., straight, zigzag, wave form, stretch stitch, knit stitch, blind hem) or others. As used herein, the term “attached” refers that sensors 620 may be sewn, knitted, embedded, or intermeshed with an elastic component 640. Sensor 620 may contain at least one thin conductive element, which in presence of a deformation, may presents a variation in an electrical characteristic (e.g., voltage, current, resistance, inductance, capacitance). The elastic component 640 may be attached to a textile portion 660 by sewing, pasting, and/or embedding.

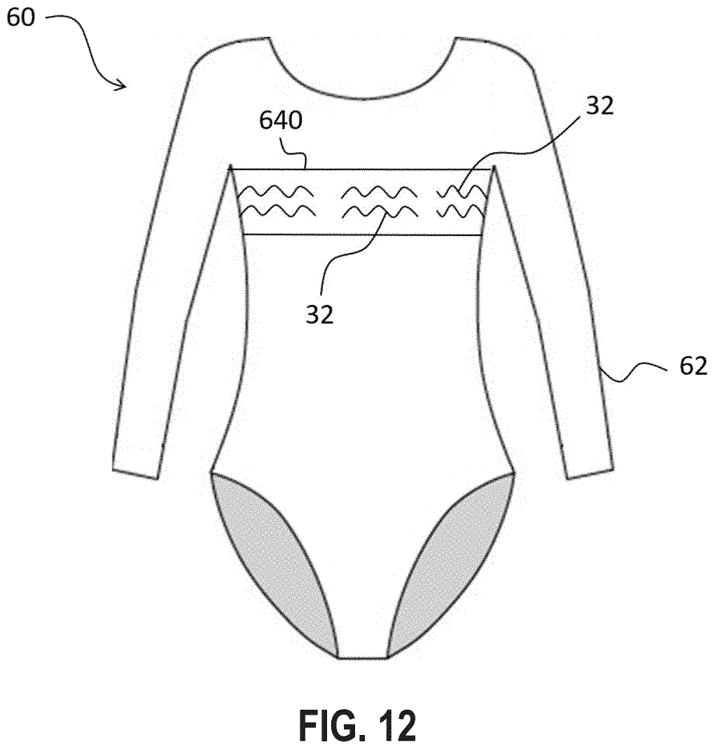

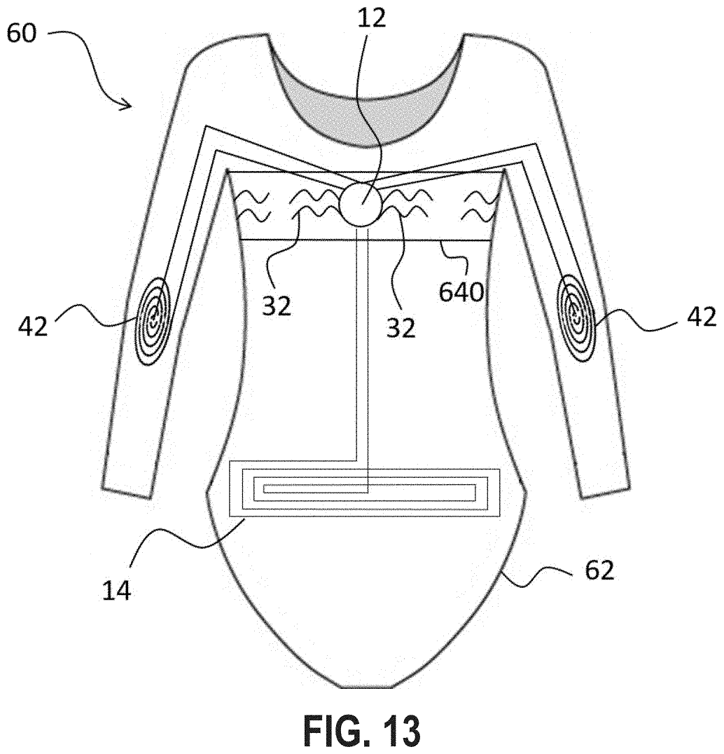

FIGS. 12 and 13 show a perspective front and rear view (FIG. 12 and FIG. 13, respectively) of a smart garment 60 for use to monitoring physiological or performance characteristics of the user. Physiological characteristics may be indicative of conditions of the user's body (e.g., respiration, heartbeat, temperature, blood pressure). Performance characteristics may be indicative of behavior of the user's body to respect to a parameter of interest (e.g., movement, position, speed, angle, etc.) Smart garment 60 may include a tight-fitting garment 62, an elastic component 640, at least one sensor 14, 32, and 42, and an electronic device 12.

Sensors 14, 32, and 42 may contain at least one thin conductive element, which in presence of a deformation, may presents a variation in an electrical characteristic (e.g., voltage, current, resistance, inductance, capacitance). Sensors 14, 32, and 42 may be attached to an elastic component 640 and/or a textile portion 62 in a flat coil configuration using basic stitches (e.g., straight, zigzag, wave form, stretch stitch, knit stitch, blind hem) or others. As used herein, the term “attached” refers that sensors 14, 32, and 42 may be sewn, knitted, embedded, or intermeshed with an elastic component 640 and/or a textile portion 62.

Electronic device 12 may contain a microcontroller to read the data transmitted by sensors 14, 32, and 42, wireless communication and batteries. Electronic device 12 may receive the data sent by sensors 14, 32, and 42 and process it in order to obtain a value of interest (e.g., angle, velocity, coordinate, distance, respiration, heart rate), as well as transmitting the data wirelessly to an external device (e.g., computer, cellphone, tablet, etc.).

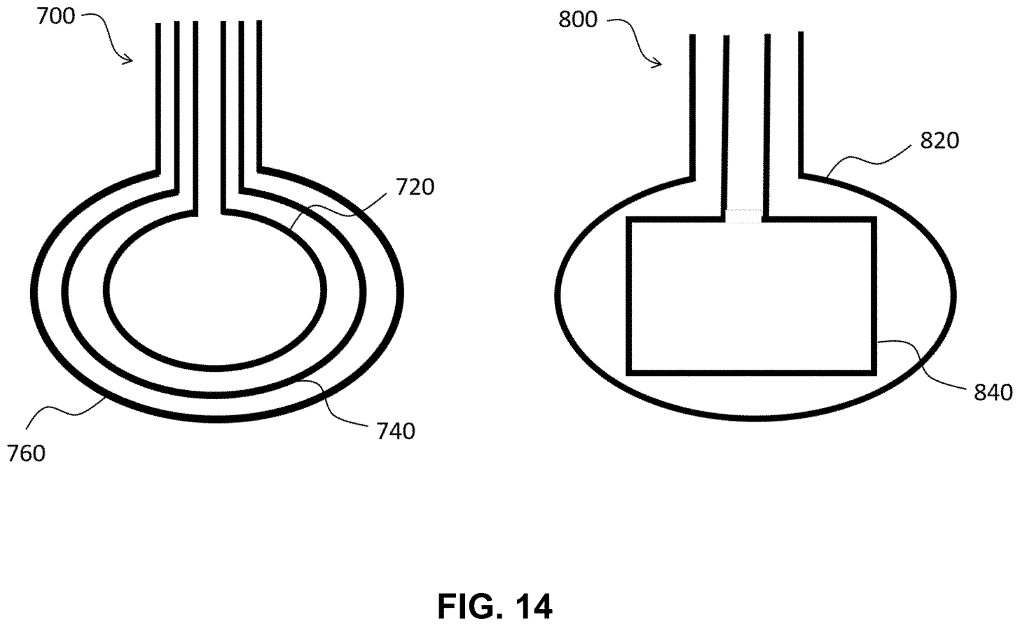

Referring now to FIG. 14, a schematic view 700 and 800 of possible configuration of concentric loops are shown, where each concentric loop may or may not have a different shape from the other concentric loops. The concentric loops can be formed by irregular, closed shapes which may have discontinuous sections, such as a circular concentric loops 720, 740, 760 and/or a circular loop 820 with an internal rectangular shape 840. The concentric loops presented by 700 and 800 are fabricated by, for example at least one sensing element 300 (shown in FIG. 8) or at least one conductive element 120 (shown in FIG. 6).

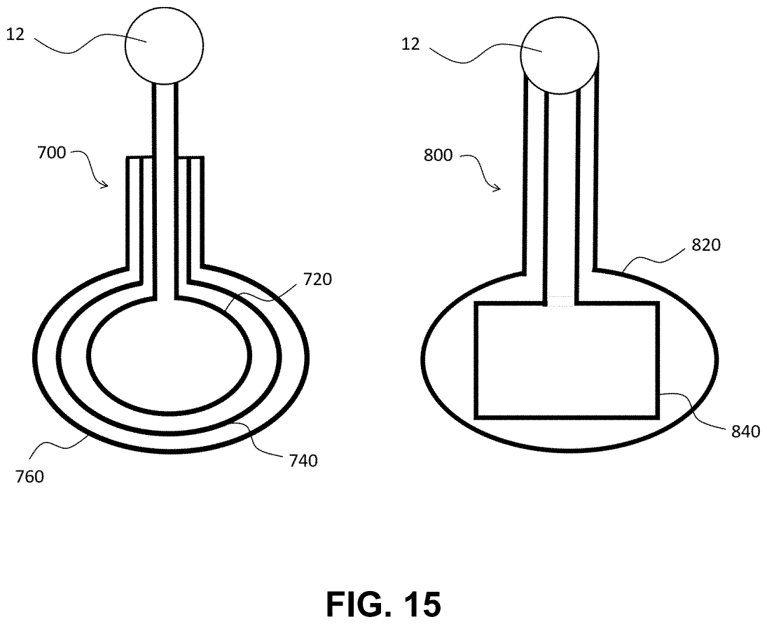

Referring now to FIG. 15, a schematic view 700 and 800 is shown of possible configurations for connecting the conductive elements to the electronic device 12. The conductive elements 720, 740 and 760 are parallel connected, as a result, mentioned conductive elements form a single loop. The conductive elements 720, 740, 760 in parallel are connected to the electronic device 12. Therefore, in example 700, the electronic device 12 will have one sensing input. The conductive elements 820 and 840 are separately connected to the electronic device 12. In 800, the input number to the electronic device 12 depends on the number of concentric loops of 800. The conductive elements 720, 740, 760, 820 and 840 as shown in FIG. 14 may be have a circular, rectangular shape, although other suitable shapes may be used as well.

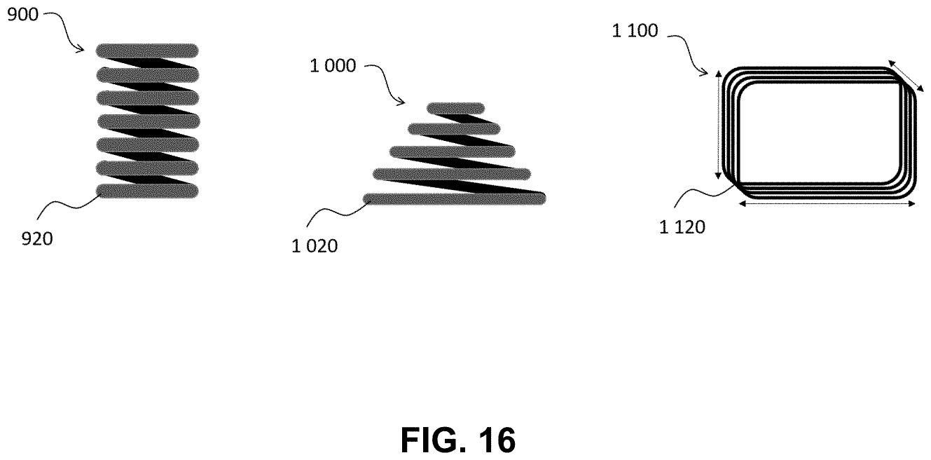

Referring now to FIG. 16, a schematic, detailed view is shown of possible 3D shape configurations of the sensing element 900, 1000 and 1100. Where the conductive element 920, 1020 or 1120 form a single loop. The loop can be formed by different shapes, such as but not limited to, spiral coil 900, conical coil 1000 and rectangular coil 1100.

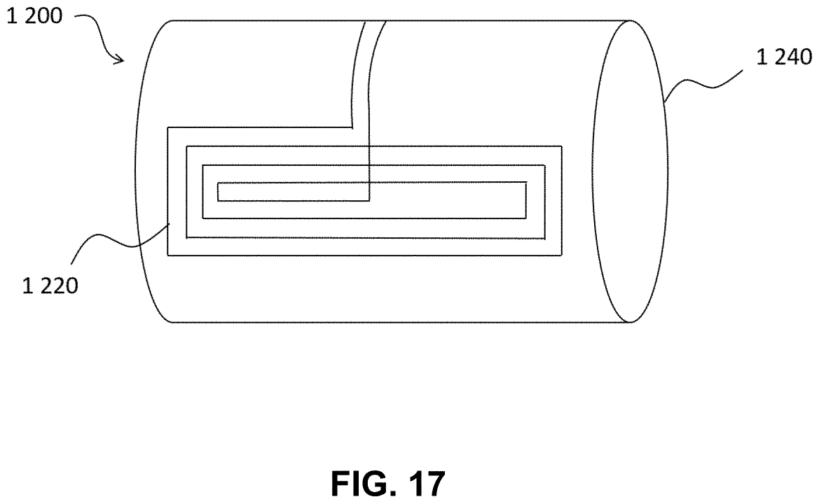

Referring now to FIG. 17, a schematic view is shown of the sensing element 1200. The sensing element 1200 may contain at least one conductive element 1220 that can be sewn, attach or embedded to a curve surface 1240. The conductive element 1220 may be stich in a flat coil configuration using a basic stich or embedded to the curve surface 1240. Sensor 1200 may consist of one conductive element, such as a thin copper wire, a conductive metallic thread or a silver coated thread. The curve surface 1240 may represent a static curve surface or a surface that is flat before deformation and curve after deformation.

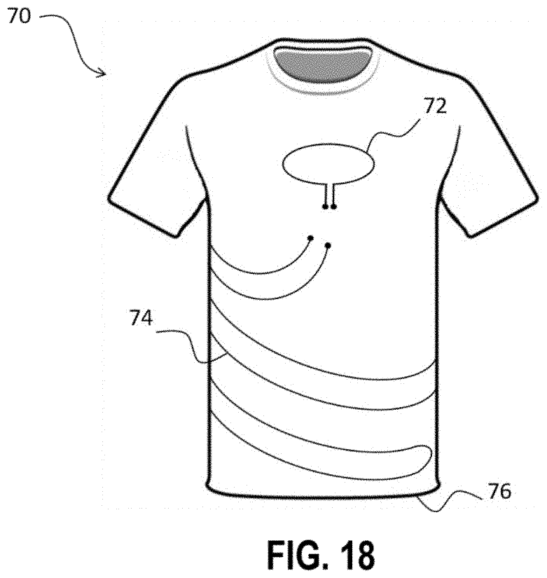

Referring now to FIG. 18, a front perspective view is shown where the surface defined by the loop only includes the textile. That is, the inner part of the loop encloses textile only. In FIG. 18, there are two loops embedded within a smart garment. A first loop 72 is relatively small whereas a second loop 74 goes around the torso. In both cases, the inner part of the loop encloses textile only. Contrast this embodiment with another where a single wire surrounds the torso of a person and where the inner loop would also enclose the torso, not only the textile.

It should be noted that textile in this context relates to a portion of a garment or cloth. Such a textile can have different shapes, be made of different materials, which could be either stretchable or not, and have ornaments or other functional elements including, but not limited to buttons, zips, brooches, stones, perforated fabric, sequins, light-emitting diodes (LEDs), digital displays, and decorative holes.

Regarding machine learning algorithms, various embodiments may be used to fully utilize the dynamic and powerful utility of machine learning and artificial intelligence. In a first embodiment, a machine learning model is developed for each single individual. In this configuration a personalized model is developed. In an alternative embodiment, a machine learning model is developed across any potential user. In this configuration one single model is developed to enable a more universal, plug-and-play use of the sensor.

In a further alternative embodiment, transfer learning is used. For example, a single model is first developed across any potential user. Such a model is then retrained with data of only one participant to make it individual-specific.

In a further alternative embodiment, a model is generated for each single configuration of the sensor. For instance, a model is trained for the spiral configuration, one model is trained for the configuration of FIG. 8, and other models are trained for other configurations.

In a further alternative embodiment, a model is generated for each location the sensor is placed. For instance, a model is trained for the knee joint, one for the elbow, one for the wrist, and other models for other body parts.

In a further alternative embodiment, a model is generated for each single application. For instance, a model is trained for gait analysis, one model is trained for good-posture, one model for monitoring breathing, and other models for other physiological, kinematic, dynamic, psychological, physical, biological or health parameters.

In a further alternative embodiment, a model is generated for each single material used in the sensor. That would include different textiles and different conductive materials.

In a further alternative embodiment, a model is generated to combine one or more parameters presented in the previous embodiments. For instance, a model could include the combination of different textiles and materials, a model could include different positions including for instance shoulder and wrist, a model could include different shapes such as spiral and single loop, a model could include a single individuals with one or multiple sensors, a model could include a single textile, different conductive materials, different sensor configurations, and a single use, such as kinematics. This embodiment includes other combinations of the previous parameters.

In a further alternative embodiment, transfer learning, which focuses on storing knowledge gained while solving one problem and applying it to a different but related problem, is used across the embodiment previously presented.

In a further alternative embodiment, a method is proposed whereby the sensor is placed adjacent to the body of a person, an electronic device reads the electrical or electromagnetic quantities associated to the sensor, variation of the electrical or electromagnetic quantities associated to the sensor are measured, and such measurements are used to estimate physiological, kinematic, dynamic, psychological, physical, biological or health parameters.

In a further alternative embodiment, the estimates for physiological, kinematic, dynamic, psychological, physical, biological or health parameters is accomplished through machine learning. Machine learning algorithms comprise supervised methods, unsupervised method, reinforcing learning, semi-supervised methods, neural networks, deep neural networks, convolution neural networks, ensembled estimators, bagging methods, decision tries, logistic regression, random forest, linear discriminant analysis, support vector machine, naïve Bayes, or K-nearest neighbors' algorithm.

In a further alternative embodiment, the sensor is placed in a position to detect movements of the lungs, air in/out the lungs or vibrations of the vocal cords. Such detection is processed to detect parameters including respiration rate, volume of air inhaled or exhaled, coughing, talking, and laughing.

In a further alternative embodiment, the sensor is placed in a position to detect movements of the heart. Such detection is processed to detect heartbeat, hear rate, opening and closing of the heart valves, blood volume and blood flow. In yet a further alternative embodiment, the sensor is placed closed to a blood vessel in close proximity to the skin to detect heart rate and blood flow.

In a further alternative embodiment, the surface defined by the loop is not crossed by any element from human body, including but not limited to, torso and limbs.

In summary, the present invention discloses a loop-based sensor, comprising at least one conductive element embedded within a textile, said at least one conductive element forming at least one loop, a textile area formed within said at least one loop, said textile area configured to be worn on the surface of the body, said textile area configured to deform said at least one conductive element with the body as it moves; and an electronic device in electrical communication with said at least one conductive element, said electronic device configured to monitor the electrical quantities of said conductive element, wherein said electrical quantities change when said at least one conductive element deforms, wherein said electronic device will detect and measure body parameters by comparing changes in electrical quantities in said at least one conductive element.

Electrical quantities of the present invention further comprise inductance and wherein said electronic device is configured to measure said inductance within said at least one conductive element. Here, the measuring of said inductance comprises measuring the oscillation frequency of said at least one conductive element. Alternatively, the electrical quantities comprise capacitance where the electronic device is configured to measure said capacitance within said at least one conductive element. Alternatively, the electrical quantities comprise resistance where the electronic device is configured to measure the opposition to the flow of electric current through said at least one conductive element.

In an alternative embodiment, the at least one conductive element is formed in a planar configuration and wherein said at least one loop forms a generally spiral configuration. Alternatively, the at least one conductive element is formed in a three-dimensional configuration and wherein said at least one loop forms a generally conical configuration. Alternatively, the at least one conductive element further comprises a plurality of conductive elements forming concentric loops. In an alternative embodiment, the at least one conductive element further comprises a plurality of discontinuous segments in electrical communication.

In an alternative embodiment, the textile further comprises an elastic, form-fitting textile, wherein said at least one conductive element is woven, knitted, or stitched into said textile, and wherein said at least one conductive element does not overlap itself.

In an alternative embodiment, the present invention further comprises a stretchable, non-conductive filament, wherein said at least one conductive element is formed around said non-conductive filament, wherein said at least one conductive element is coiled around said filament.

In an alternative embodiment, the textile further comprises a patch, wherein said patch is affixed to a garment for monitoring body parameters. Here, the patch further comprises a wireless communication device, wherein said wireless communication device is configured to communicate any detected movement and physical parameters to an external device.

In an alternative embodiment, the body parameters comprise body movement and other bodily functions. Body parameters further comprise limb movements, body flexion, body extension, body expansion, body contraction, joint rotations, joint angles, joint position, posture, gait, step length, stride length, cadence, speed, foot angle, hip angle, knee angle, gait phase, body height, body weight, body mass index, movements of the lungs, air in/out the lungs, vibrations of the vocal cords, respiration rate, volume of air inhaled or exhaled, coughing, talking, laughing, movements of the heart, heartbeat, opening and closing of the heart valves, blood volume and blood flow, deformation of blood vessels, heart rate, blood flow, and temperature. The body parameters are used to estimate health parameters, said health parameters comprising physiological, kinematic, dynamic, psychological, physical, biological, or other health parameters.

In an alternative embodiment, the body parameters further comprise machine learning algorithms configured to monitor and compare said body parameters, said machine learning further comprising supervised methods, unsupervised method, reinforcement learning, transfer learning, encoders, decoders, semi-supervised methods, neural networks, deep neural networks, convolution neural networks, ensembled estimators, bagging methods, decision tries, logistic regression, random forest, linear discriminant analysis, support vector machine, naïve Bayes, and K-nearest neighbors algorithm.

A method of monitoring bodily physical parameters comprises embedding a conductive element within an elastic, form-fitting textile, positioning said textile adjacent to the body, wherein said textile and said conductive element are configured to deform with the body as it moves, measuring variations of electrical or electromagnetic quantities within said conductive element as it deforms with the body, and estimating physiological, kinematic, dynamic, psychological, physical, biological or health parameters based upon said variations of electrical or electromagnetic quantities.

In an alternative embodiment, the estimation of physiological, kinematic, dynamic, psychological, physical, biological or health parameters further comprises machine learning estimation comprising supervised methods, unsupervised method, reinforcement learning, transfer learning, encoders, decoders, semi-supervised methods, neural networks, deep neural networks, convolution neural networks, ensembled estimators, bagging methods, decision tries, logistic regression, random forest, linear discriminant analysis, support vector machine, naïve Bayes, and K-nearest neighbors algorithm.

While the above description contains specific details regarding certain elements, sizes, and other teachings, it is understood that embodiments of the invention or any combination of them may be practiced without these specific details. Specifically, although certain materials and shapes are designated in the above embodiments, any suitable materials or shapes may be used. These details should not be construed as limitations on the scope of any embodiment, but merely as exemplifications of the presently preferred embodiments. In other instances, well known structures, elements, and techniques have not been shown to clearly explain the details of the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

What is claimed is:

1. A loop-based sensor, comprising:
at least one conductive element embedded within a textile, said at least one conductive element forming at least one loop;
a textile area formed within said at least one loop, said textile area configured to be worn on the surface of the body, said textile area configured to deform said at least one conductive element with the body as it moves; and
an electronic device in electrical communication with said at least one conductive element, said electronic device configured to monitor the electrical quantities of said conductive element, wherein said electrical quantities change when said at least one conductive element deforms;
wherein said electronic device will detect and measure body parameters by comparing changes in electrical quantities in said at least one conductive element.

2. The sensor of claim 1, wherein said electrical quantities comprise inductance and wherein said electronic device is configured to measure said inductance within said at least one conductive element.

3. The sensor of claim 2, wherein said measuring of said inductance comprises measuring the oscillation frequency of said at least one conductive element.

4. The sensor of claim 1, wherein said electrical quantities comprise capacitance and wherein said electronic device is configured to measure said capacitance within said at least one conductive element.

5. The sensor of claim 1, wherein said electrical quantities comprise resistance and wherein said electronic device is configured to measure the opposition to the flow of electric current through said at least one conductive element.

6. The sensor of claim 1, wherein said at least one conductive element is formed in a planar configuration and wherein said at least one loop forms a generally spiral configuration.

7. The sensor of claim 1, wherein said at least one conductive element is formed in a three-dimensional configuration.

8. The sensor of claim 7, wherein said three-dimensional configuration forms a generally conical configuration.

9. The sensor of claim 1, wherein said at least one conductive element further comprises a plurality of conductive elements forming concentric loops.

10. The sensor of claim 1, wherein said at least one conductive element further comprises a plurality of discontinuous segments.

11. The sensor of claim 1, wherein said textile further comprise an elastic, form-fitting textile, wherein said at least one conductive element is stitched into said textile, and wherein said at least one conductive element does not overlap itself.

12. The sensor of claim 1, wherein said textile further comprises a stretchable, non-conductive filament, wherein said at least one conductive element is formed around said non-conductive filament, wherein said at least one conductive element has a generally coiled shape.

13. The sensor of claim 1, wherein said textile further comprises a patch, wherein said patch is affixed to a garment for monitoring body parameters.

14. The sensor of claim 13, wherein said patch further comprises a wireless communication device, wherein said wireless communication device is configured to communicate any detected movement and physical parameters to an external device.

15. The sensor of claim 1, wherein said body parameters further comprise limb movements, body flexion, body extension, body expansion, body contraction, joint rotations, joint angles, joint position, posture, gait, step length, stride length, cadence, speed, foot angle, hip angle, knee angle, gait phase, body height, body weight, body mass index, movements of the lungs, air in/out the lungs, vibrations of the vocal cords, respiration rate, volume of air inhaled or exhaled, coughing, talking, laughing, movements of the heart, heartbeat, opening and closing of the heart valves, blood volume and blood flow, deformation of blood vessels, heart rate, blood flow, and temperature.

16. The sensor of claim 1, wherein said body parameters are used to estimate health parameters, said health parameters comprising physiological, kinematic, dynamic, psychological, physical, biological, or other health parameters.

17. The sensor of claim 1, further comprising machine learning algorithms configured to monitor and compare said body parameters, said machine learning further comprising supervised methods, unsupervised method, reinforcement learning, transfer learning, encoders, decoders, semi-supervised methods, neural networks, deep neural networks, convolution neural networks, ensembled estimators, bagging methods, decision tries, logistic regression, random forest, linear discriminant analysis, support vector machine, naïve Bayes, and K-nearest neighbors algorithm.

18. A body sensor, comprising:
an elastic, form-fitting garment configured to be worn on the body, said garment configured to deform with the body as it moves;
a conductive element embedded within said garment, said conductive element configured to deform with said garment as the body moves; and
an electronic device in electrical communication with said conductive element, said electronic device configured to measuring the oscillation frequency of said at least one conductive element, wherein said oscillation frequency changes when said conductive element deforms;
wherein said body sensor will measure movements of the body by comparing changes in inductance in said conductive element.

19. A method of monitoring body movement, said method comprising:
embedding a conductive element within an elastic, form-fitting textile, said conductive element forming a loop, wherein the area within the loop forms a textile area;
positioning said textile area adjacent to the body, wherein said textile area and said conductive element are configured to deform with the body as it moves;
measuring variations of electrical or electromagnetic quantities within said conductive element as it deforms with the body; and
estimating physiological, kinematic, dynamic, psychological, physical, biological or health parameters based upon said variations of electrical or electromagnetic quantities.

20. The method of claim 19, wherein said conductive element is spun around a non-conductive fabric.
